(19) United States
(12) Patent Application Publication
(10) Pub. No.: US 2024/0138776 A1
(43) Pub. Date: May 2, 2024

(54) WEARABLE NON-INVASIVE BLOOD GLUCOSE MONITORING SYSTEM

(71) Applicant: QATAR UNIVERSITY, Doha (QA)

(72) Inventors: Mazun ALSHAHWANI, Doha (QA); Noora AL BORDENI, Doha (QA); Fatima AL-KAABI, Doha (QA); Sara AL-MOHANNADI, Doha (QA); Khalid ABUALSAUD, Doha (QA); Muhammad E.H. CHOWDHURY, Doha (QA); Elias YAACOUB, Doha (QA); Nizar ZORBA, Doha (QA)

(21) Appl. No.: 18/386,189

(22) Filed: Nov. 1, 2023

Related U.S. Application Data
(60) Provisional application No. 63/421,416, filed on Nov. 1, 2022.

(51) Int. Cl. A61B 5/00; A61B 5/0205; A61B 5/021; A61B 5/024

(52) U.S. Cl. CPC A61B 5/7278; A61B 5/0205; A61B 5/021; A61B 5/02416; A61B 5/7267; A61B 5/7275; A61B 5/746; A61B 5/7475

(57) ABSTRACT

Systems, methods, apparatuses, and computer program products for non-invasive glucose monitoring. A method may include continuously capturing a photoplethysmography signal of a patient in real-time. The method may also include inputting a blood pressure measurement, demographic information, and the photoplethysmography signal into a mobile application. The method may further include transmitting, as input information, the blood pressure measurement, the demographic information, and the photoplethysmography signal to a cloud server. In addition, the method may include generating, based on the input information, a glucose prediction of the patient.

### CROSS-REFERENCE TO RELATED APPLICATIONS

This application claims priority from U.S. provisional patent application No. 63/421,416 filed on Nov. 1, 2022. The contents of this earlier filed application are hereby incorporated by reference in their entirety.

### FIELD

Some example embodiments may generally relate to technologies in biological monitoring systems. More specifically, certain example embodiments may relate to a wearable non-invasive blood glucose monitoring system.

### BACKGROUND

Diabetes is a widespread disease, and studies have reported that there has been a significant increase in the incidence of diabetes, which is a pathological condition in which the body is unable to utilize glucose (body sugar) efficiently. This leads to the accumulation of sugar in the blood, and the patient develops diabetes if the body does not produce enough insulin, or if it is resistant to it.

There are many types of diabetes, of which the most critical is Type-1 (T1D), which results from the destruction of the cells that produce insulin. This means that the pancreas stops producing insulin permanently, and when infected with it, the patient's life depends on insulin injections. As for Type-2 (T2D), the pancreatic production of insulin is weak, and it is less dangerous than the first type, and it can be prevented. Usually, it results from unhealthy behaviors and lifestyles. In addition, some other types of diabetes may include those that appear in some women during pregnancy, however, they are rare.

Diabetes poses serious health challenges in various countries around the world. For this reason, strategies have been developed that aimed at formulating an advanced vision for health care for diabetic patients in the future to enhance the state of health and quality of life by working to achieve the goal of preventing diabetes. If not all required steps to control diabetes are followed, the rate of diabetes may double in the age range of 35-60 years by two and a half times by 2045.

Modern glucose devices of Type-2 (T2D) do not have a continuous reading of blood glucose; however, they should be scanned on the arm between intermittent periods to take blood sugar data. Regardless of its ability to continuously measure glucose, it still needs finger pricking at least twice a day for calibration. Continuous glucose monitoring (CGM) is not as robust as blood glucose meters (BGMs) while more costly and most importantly, for CGMs, it is required to insert the sensor under the skin of the subject (e.g., abdomen, thighs, upper arms, etc.) which is even more uncomfortable and painful than BGMs. So, regardless of the drawbacks of using BGMs, due to the lack of robust non-invasive alternatives, most clinics around the world still fully or partially rely on BGMs. Based on the growing demand for glucose monitoring devices, a reliable, non-invasive, and easy-to-use alternative to BGMs can quickly capture some of its market shares.

Some recent studies used noninvasively collected Photoplethysmography (PPG) raw signals or extracted features as input for machine learning (ML) algorithms to estimate blood glucose level (BGL). However, the performance of these systems is either not up to the level of acceptance yet, or the dataset used for validation is very small. Moreover, these systems are relying on PPG signals alone and there are several other physiological data and demographic factors can lead to change in BGL. However, from these studies, it can be ascertained that PPG signals have a great promise in estimating blood glucose.

In view of the above, there is a need for a reliable blood glucose measurement from PPG signals, blood pressure data, and demographic information.

### SUMMARY

Some embodiments may be directed to a method. The method may include continuously capturing a photoplethysmography signal of a patient in real-time. The method may also include inputting a blood pressure measurement, demographic information, and the photoplethysmography signal into a mobile application. The method may further include transmitting, as input information, the blood pressure measurement, the demographic information, and the photoplethysmography signal to a cloud server. In addition, the method may include generating, based on the input information, a glucose prediction of the patient.

Other embodiments may be directed to a glucose monitoring system. The glucose monitoring system may include a monitoring device including a biological sensor and microcontroller configured to continuously capture a photoplethysmography signal of a patient in real-time. The blood glucose monitoring system may also include a user equipment in communication with the monitoring device, wherein the user equipment is configured to receive, as input information, a blood pressure measurement, demographic information, and the photoplethysmography signal. The blood glucose monitoring system may further include a server in communication with the user equipment, wherein the server is configured to generate, based on the input information, a glucose prediction of the patient.

Other example embodiments may be directed to an apparatus. The apparatus may include means for continuously capturing a photoplethysmography signal of a patient in real-time. The apparatus may also include means for inputting a blood pressure measurement, demographic information, and the photoplethysmography signal into a mobile application. The apparatus may further include means for transmitting, as input information, the blood pressure measurement, the demographic information, and the photoplethysmography signal to a cloud server. In addition, the apparatus may include means for generating, based on the input information, a glucose prediction of the patient.

In accordance with other example embodiments, a non-transitory computer readable medium may be encoded with instructions that may, when executed in hardware, perform a method. The method may include continuously capturing a photoplethysmography signal of a patient in real-time. The method may also include inputting a blood pressure measurement, demographic information, and the photoplethysmography signal into a mobile application. The method may further include transmitting, as input information, the blood pressure measurement, the demographic information, and the photoplethysmography signal to a cloud server. In addition, the method may include generating, based on the input information, a glucose prediction of the patient.

### BRIEF DESCRIPTION OF THE DRAWINGS

The accompanying drawings, which are included to provide a further understanding of the invention and are incorporated in and constitute a part of this specification, illustrate preferred embodiments of the invention and together with the detail description serve to explain the principles of the invention. In the drawings:

FIG. 1 illustrates an example architecture of a method, according to certain example embodiments.

FIG. 2 illustrates an example graphical comparisons of differences in photoplethysmography (PPG) signals for different glucose levels, according to certain example embodiments.

FIG. 3(a) illustrates an example of hardware components of a system during a work mode, according to certain example embodiments.

FIG. 3(b) illustrates an example of hardware components of the system during charging mode, according to certain example embodiments.

FIG. 4 illustrates an example size of a continuous glucose monitoring (CGM) device, according to certain example embodiments.

FIG. 5A illustrates an example exploded view of the CGM device, according to certain example embodiments.

FIG. 5B illustrates an example of another exploded view of the CGM device, according to certain example embodiments.

FIG. 5C illustrates an example of a further exploded view of the CGM device, according to certain example embodiments.

FIG. 6A illustrates an example an assembly of the CGM device, according to certain example embodiments.

FIG. 6B illustrates another example of the assembly of the CGM device, according to certain example embodiments.

FIG. 6C illustrates a further example of the assembly of the CGM device, according to certain example embodiments.

FIG. 6D illustrates a further example of the assembly of the CGM device, according to certain example embodiments.

FIG. 7 illustrates an example CGM complete device, according to certain example embodiments.

FIG. 8 illustrates an example PPG signal by the CGM device, according to certain example embodiments.

FIG. 9 illustrates an example user interface (UI), according to certain example embodiments.

FIG. 10 illustrates an example flow diagram of a method, according to certain example embodiments.

FIG. 11 illustrates an apparatus according to certain example embodiments.

### DETAILED DESCRIPTION

It will be readily understood that the components of certain example embodiments, as generally described and illustrated in the figures herein, may be arranged and designed in a wide variety of different configurations. The following is a detailed description of some embodiments of systems, methods, apparatuses, and/or computer program products for a wearable non-invasive blood glucose monitoring system.

The features, structures, or characteristics of example embodiments described throughout this specification may be combined in any suitable manner in one or more example embodiments. For example, the usage of the phrases “certain embodiments,” “an example embodiment,” “some embodiments,” or other similar language, throughout this specification refers to the fact that a particular feature, structure, or characteristic described in connection with an embodiment may be included in at least one embodiment. Thus, appearances of the phrases “in certain embodiments,” “an example embodiment,” “in some embodiments,” “in other embodiments,” or other similar language, throughout this specification do not necessarily all refer to the same group of embodiments, and the described features, structures, or characteristics may be combined in any suitable manner in one or more example embodiments.

Additionally, if desired, the different functions or steps discussed below may be performed in a different order and/or concurrently with each other. Furthermore, if desired, one or more of the described functions or steps may be optional or may be combined. As such, the following description should be considered as merely illustrative of the principles and teachings of certain example embodiments, and not in limitation thereof.

Certain example embodiments may provide a real-time, continuous, non-invasive, and reliable blood glucose measurement from PPG signals, blood pressure data, and demographic information. The BGM system of certain example embodiments may continuously capture the PPG signal from the wrist in real-time, and send the PPG signal to a smart phone application over BLUETOOTH®. A patient may input his/her pulse, systolic and diastolic blood pressure along with demographic information (e.g., age, race, gender, body mass index (BMI), etc.) to the mobile application, and the PPG signal along with other information may be sent to a cloud server every 10 seconds. The server may report the estimated glucose value to the mobile application based on the information received from the mobile application.

According to certain example embodiments, the CGM device may continuously measure blood sugar, and the mobile application may notify the patient in case of a significant increase of blood sugar that exceeds the limit of the normal sugar level, or in case it drops below the limit. Current glucose testing methods continue to rely on intrusive techniques that are unpleasant and increase the risk of infection, whereas the system according to certain example embodiments may be non-invasive and capable of measuring blood glucose reliably due to the incorporation of blood pressure and demographic data along with PPG signal which is not used in any system earlier.

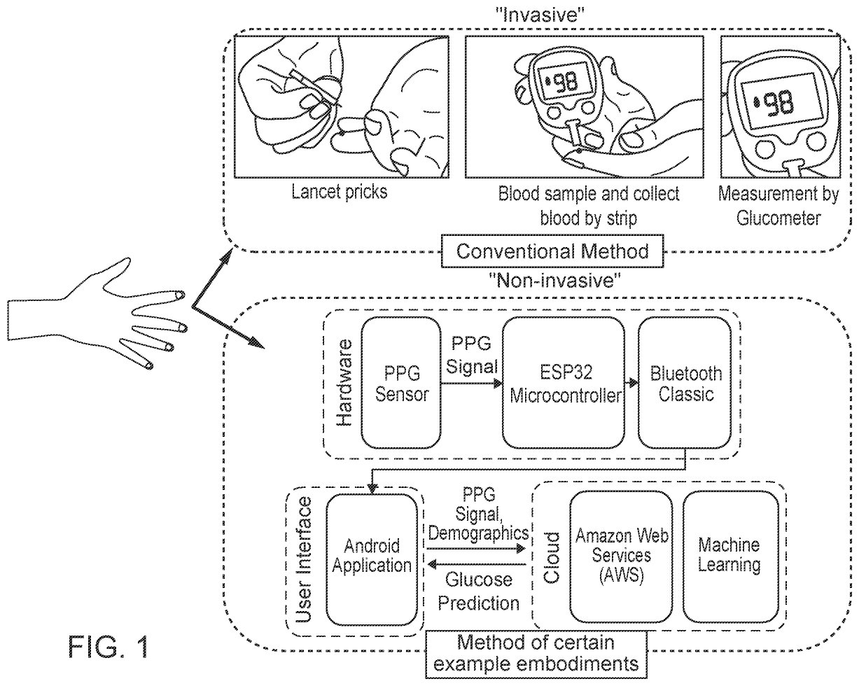

FIG. 1 illustrates an example methodology with respect to the traditional methodology, according to certain example embodiments. As illustrated in FIG. 1, the wearable system of certain example embodiments may be attached to the wrist of a patient for comfortable capturing of a PPG signal, and the wearable system can be used all the time whereas the software part of the system may include integrating a mobile application and backend server for continuous monitoring (e.g., every 10 seconds update, or time period below or above 10 seconds).

As illustrated in FIG. 1, the system of certain example embodiments may include a PPG or lightweight pulse sensor for capturing PPG signals. The system may also include a tiny microcontroller (e.g., TinyPICO) to control the circuitry of the system. The system (e.g., CGM device) may further include an ON/OFF switch to manually turn on and off the device, and a small lithium-ion (Li-ion) battery to power the system.

As further illustrated in FIG. 1, the microcontroller may be integrated with BLUETOOTH® capability, enabling the system to connect to a user interface of a mobile application (e.g., software application) implemented on a computing device. The mobile application may transmit the PPG signal and demographics information of the patient to a cloud server that uses ML to generate a glucose prediction. The cloud server may then transmit the glucose prediction to the mobile application, which may inform the user of the system concerning the glucose prediction.

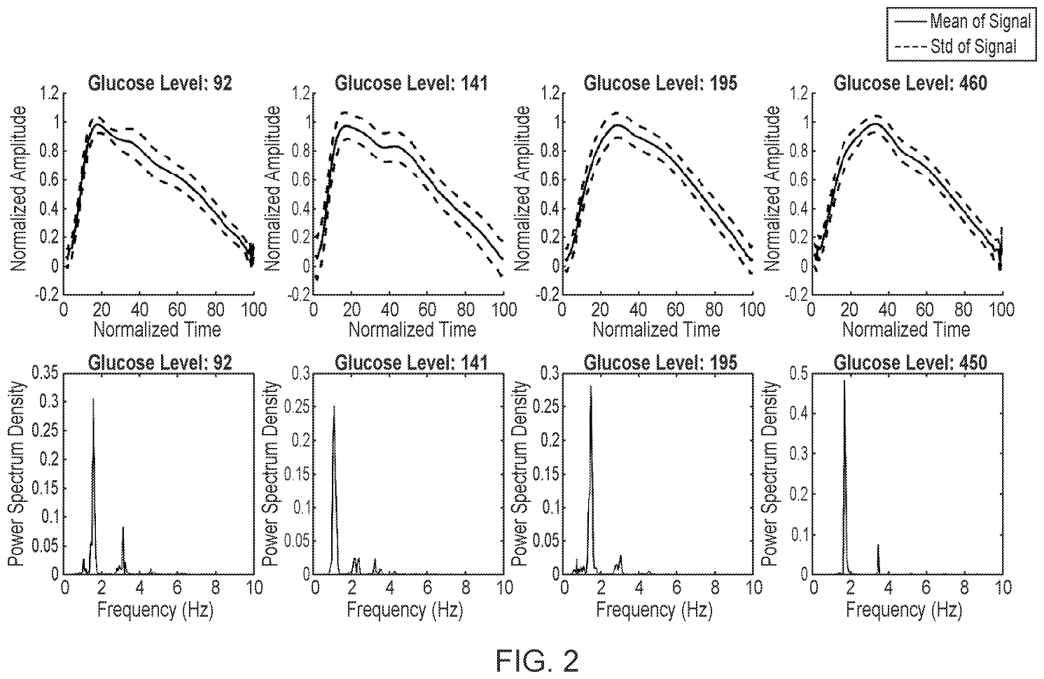

FIG. 2 illustrates an example difference in morphology PPG signal for different glucose levels, according to certain example embodiments. According to certain example embodiments, it may be necessary to see whether there is any change in PPG morphology across various glucose levels. Each beat may be separated and then an average morphology of the signal may be plotted in a time-normalized and amplitude-normalized fashion. The mean morphology along with standard deviation is illustrated in FIG. 2. For example, at lower levels of glucose, the average morphology shows more prominence in notch areas. This disappears at higher glucose levels. Furthermore, systolic peak time may be larger with higher blood glucose levels. In FIG. 2, the power spectrum density of the signals is also shown at the bottom, and there are only 2 spikes with a glucose level of 450 mg/dL compared to the other three signals.

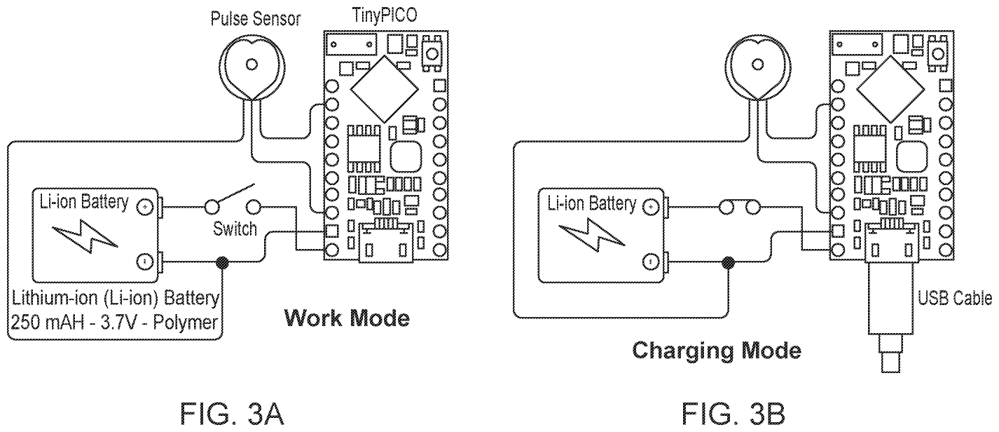

According to certain example embodiments, a TinyPICO may be selected as the microcontroller for controlling the circuit due to it being a small, fully featured ESP32 development board. Contained in a package as small as 18mmu32 mm, the TinyPICO may efficiently utilize ESP32's 240 MHz dual core while having Bluetooth Low Energy (BLE 4.2) and Wi-Fi connectivity options. A feature of TinyPICO may be its ability to efficiently manage Lithium-Polymer (LIPO) or Li-ion batteries using its onboard 3.3V Low-Dropout (LDO) voltage regulator. Thus, a Li-ion battery can be charged and discharged by the microcontroller alone without any need for external circuitry which greatly reduced the prototype size and weight. As illustrated in FIG. 3(a), during work mode (i.e., while being used), the battery supplies power to the circuit which can be turned on and off. On the other hand, as illustrated in FIG. 3(b), when the circuit is connected to a central power supply (e.g., personal computer, power bank, etc.), the battery gets charged instead when the switch is on. The charging process can be stopped by turning the switch off. The pulse sensor may utilize a supply of 5V to run while the battery can only supply 3.7V. The onboard LDO may enable feeding the pulse sensor without the use of an external power boost converter, thus reducing the price while improving the portability and compactness of the device. There may be various sizes and weights of Li-ion batteries available in the market, and their size and/or weight may directly correlate to their charge storage capacity (milliampere-hour or mAh) while maintaining a fixed voltage output of 3.7V. For the device of certain example embodiments, a tiny 3.7V-250 mAh Li-ion battery that may last up to 24 hours, and having similar weight and size as the TinyPICO may be selected to power the circuitry.

According to certain example embodiments, a power switch may be incorporated into the circuit of the non-invasive glucose monitoring device. The power switch may enable the ability to manually turn the device on or off to prevent the battery charger from drying out. As described herein, the PPG signals may be collected by the CGM device which may be wirelessly transmitted to an application implemented in a computing device using a BLUETOOTH® protocol supported by the microcontroller. The application may receive the signal and send it to a remote server uploaded on a web services server. According to certain example embodiments, the demographic and blood pressure information of the user may be updated in the mobile application by the user. Additionally, the remote server may analyze the signal and extract the value of glucose.

In certain example embodiments, the non-invasive glucose monitoring device (e.g., CGM device) may have unique design aspects. For example, the glucose monitoring device may be designed and manufactured such that it is painless while in use. In some example embodiments, the CGM device may display the blood sugar level of the user without causing pain to the user. The CGM device may be non-invasive, and may not require the use of a needle or the taking of a blood sample in order to measure the blood sugar level of the user.

In other example embodiments, the CGM device may use a special technology such as, for example, PPG signaling. Since numerous features may be extracted from the PPG signal, it may be useful in a variety of diseases. However, PPG alone may not be a reliable source of glucose estimation. As such, in certain example embodiments, additional physiological and demographic information may be added/implemented with the CGM device to make the entire system robust and unique.

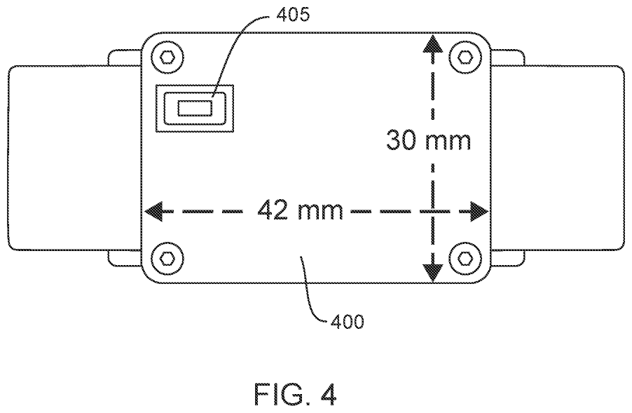

According to some example embodiments, the implementation of a non-invasive technology may have an impact on cultural awareness by altering people's perceptions of common equipment for detecting blood glucose using a blood sample. According to other example embodiments, the CGM device may not require the patient to change a needle and/or strips on a regular basis. Further, in certain example embodiments, the CGM may be designed to be aesthetically pleasing in appearance, with no indication that it is designed for diabetic patients, so that the patient does not feel embarrassed while showing it to others as the design resembles a watch. Additionally, the CGM device of certain example embodiments may be convenient and comfortable to wear. As a result, the device may be compact and may be worn on the wrist. As illustrated in FIG. 4, the size of the CGM device 400 of certain example embodiments may be 30 mm×42 mm, and the CGM device may include a ON/OFF switch 405. Furthermore, the CGM device may be light on the user's hand to provide comfort. To achieve this, for example, the CGM device may be made up of light and compact components, resulting in a device that weighs approximately 17 grams.

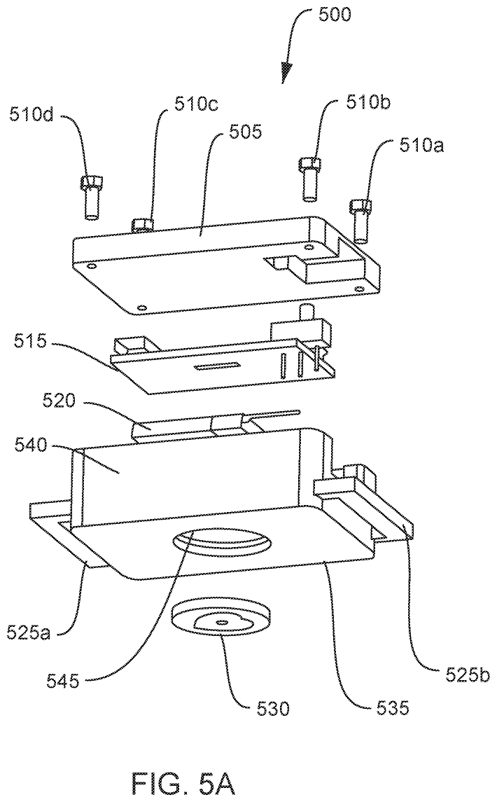

FIGS. 5A-5C illustrate example exploded views of the CGM device 500, according to certain example embodiments. As illustrated in FIGS. 5A-5C, the CGM device 500 may be made up of an outer plate 505 attached to a casing 540 via fasteners (e.g., screws or bolts) 510a-510d. The casing 540 may house the microcontroller board 515, and the Li-ion battery 520 which powers the CGM device 500. As illustrated in FIGS. 5A-5C, the casing 540 may include a plate 535 disposed opposite of the outer plate 505. The plate 535 may include a cutout 545 large enough to house a PPG sensor 530. The CGM device 500 may also include two extending portions 525a, 525b, where each extending portion 525a, 525b devices an opening sufficiently large enough to allow a strap (not shown) to pass through and fastened to the CGM device 500. The strap may enable the CGM device 500 to be worn by the user at, for example, the user's wrist.

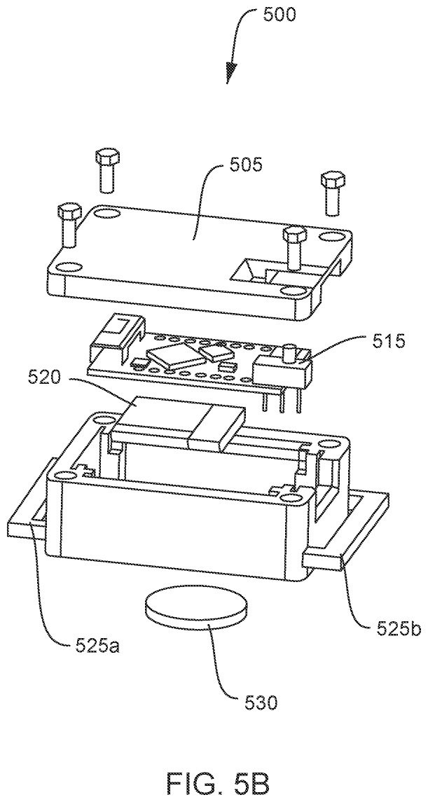

FIGS. 6A-6D illustrate an example of an assembly of the CGM device, according to certain example embodiments. In particular, FIG. 6A illustrates the CGM device which may include a casing 605 and a plate 625. As illustrated in FIG. 6D plate 625 may include an opening that houses the PPG sensor 630. The CGM device may also include straps 600 for attaching to a user. As illustrated in FIGS. 6B and 6C, the CGM device may include a microcontroller 615 covered by an outer plate 610. The outer plate 610 may be fastened to the casing 605 via fasteners 620 (e.g., screws or bolts) through corresponding holes in the outer plate 610 and casing 605.

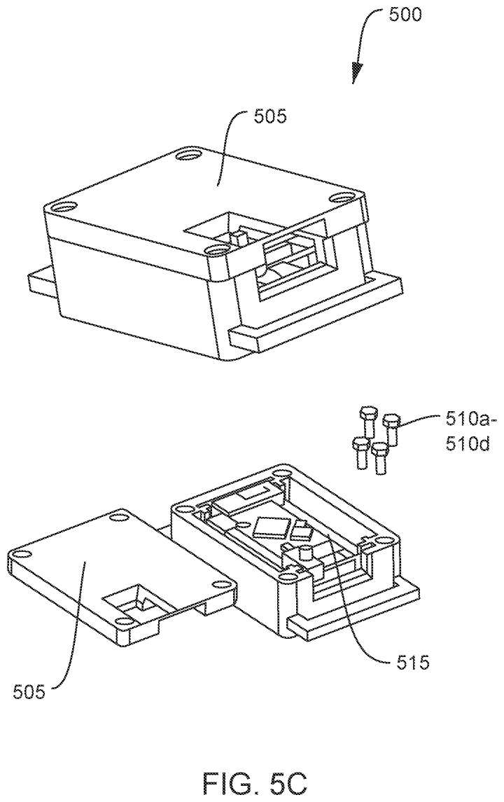

FIG. 7 illustrates a complete CGM device 700, according to certain example embodiments. In some example embodiments, after a successful connection, the microcontroller may be programmed using Arduino IDE. The position of the PPG sensor may be adjusted in the wrist as shown in FIG. 7. Then, immediately, the PPG signal may be sent to the application when the device executing the mobile application is “ON”. The mobile application may be able to accommodate 10s' data and the send the data to the remote server to estimate the glucose level and display it to the patient in the mobile application.

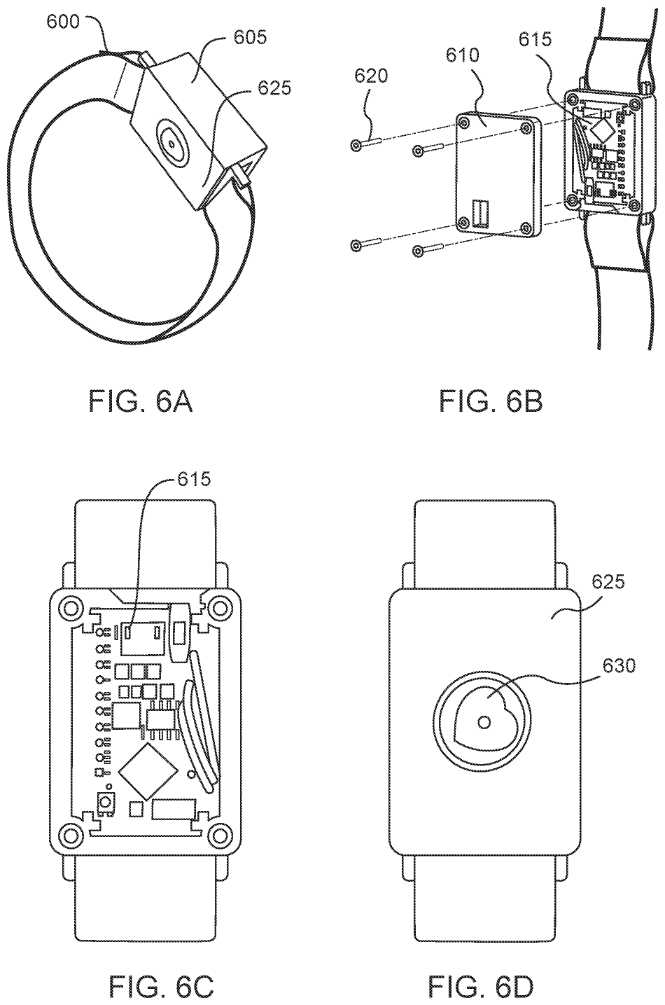

FIG. 8 illustrates an example PPG signal by the CGM device, according to certain example embodiments. In particular, FIG. 8 illustrates the test phase of the CGM device with a computer to see the reliable acquisition of the PPG signals to the computer, which may be subsequently sent to a mobile application via BLUETOOTH®. In certain example embodiments, an application (e.g., Android application) may be developed to make the algorithm accessible to the consumer. The purpose of the application may be to provide an intuitive way for the user so they can make full use of the proposed glucose level prediction algorithm. Several platforms may be considered for developing the software. For instance, the Android platform may be selected for its ability to run on mobile devices, and for its accessibility on various readily accessible mobile devices.

According to certain example embodiments, the mobile application may collect the data from the PPG sensor wirelessly and then send it to the server where it may be processed using the ML model to provide the glucose prediction. Further, a mobile application may be developed for the Android operating system, and it may have support for devices running Android version 5.0 (Lollipop) or newer. According to some example embodiments, the application may be written in the Kotlin programming language instead of the traditional Java programming language to support more advanced features and architectural patterns. The basic functionality of the application can be divided into two part.

The first part may relate to BLUETOOTH® communication. For example, to wirelessly transfer the data from the PPG sensor, certain example embodiments may use a BLUETOOTH® classic protocol. To support continuous data transmission at a high sampling rate, classical BLUETOOTH® may be selected over the BLUETOOTH® low energy (BLE) protocol. When connected to the sensor, the application may receive the PPG data at a 100 samples/sec rate. Upon receiving the data, it may be stored in a temporary buffer of the computing device (e.g., mobile device) executing the application. The data may be stored with a length of 2048 samples. Once the buffer is full, the data may then be handed over to the network module of the application, which may handle the communication with the server.

The second part may relate to network communication. For example, the application may handle the traffic between the backend server and the application. At first, the PPG data along with the demographic information of the user may be sent to the server via an HTTP POST request. The body of the request may include all the outgoing data in JavaScript Object Notation (JSON) format. Once the request is received by the server, it may transfer the data to the ML model to calculate the glucose prediction. Once the predicted value is available, the server may respond with a JSON object containing the glucose prediction. The application may then receive the response from the server and displays it to the user. If anything goes wrong in the in-between process (e.g., network error, invalid data, etc.), the server may throw an error which may then surfaced to the user.

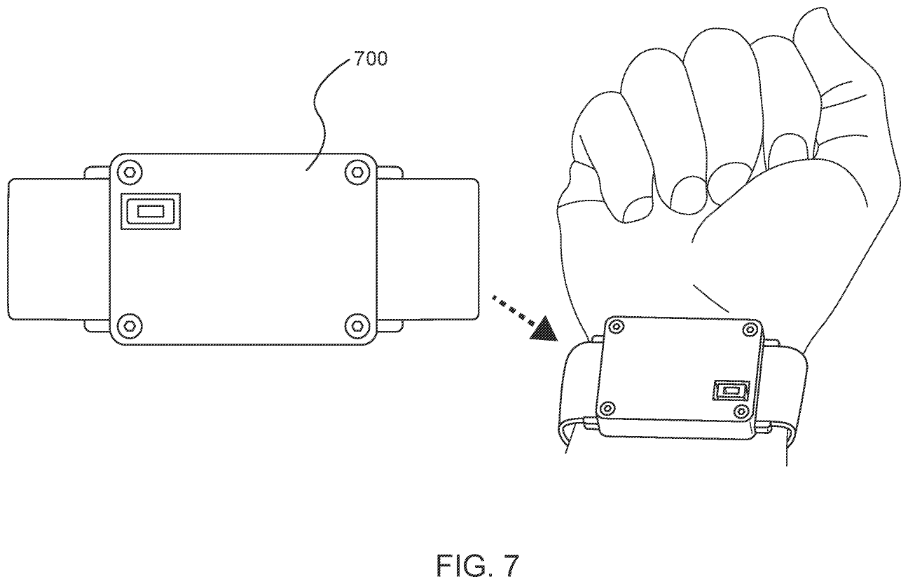

FIG. 9 illustrates an example user interface (UI) of different screens of the application, and basic instructions for using the application, according to certain example embodiments. According to certain example embodiments, certain instructions may be followed while using the application for the first time. The instructions may include, for example, the user needs to pair the PPG device to their smartphone using the BLUETOOTH® connectivity section of the device. Once the pairing process is done, the user can open the application. On first-time use, the application may ask for the required permissions. At that point, the user may be requested to approve all the permissions in this part otherwise the application will not function. After granting the permission, the user may be prompted to turn on the BLUETOOTH® if not enabled already.

Further, the user may be presented with a list of paired devices. If the desired device is not on the list, the user may click a “Refresh Device Button” which will refresh the list. Upon clicking on the button for the desired device, a BLUETOOTH® connection process will take place. Upon successful connection, the list may become invisible, and the user may be presented with a form to fill up the demographic information. Once the demographic information is correctly filled up, the user can click on the “Start” button which will start sending the data to the server. Initially, the CGM device may wait for the PPG buffer to fill up and after that, the data will be transmitted every 10 seconds. Once the response from the server is available, it may be displayed in the UI (e.g., in blue color) at the top of the screen. The glucose prediction may be updated automatically as new data are transferred to the server. After using the application, the user can close the application by clicking back on the navigation menu which will close the BLUETOOTH® socket and server-side communication.

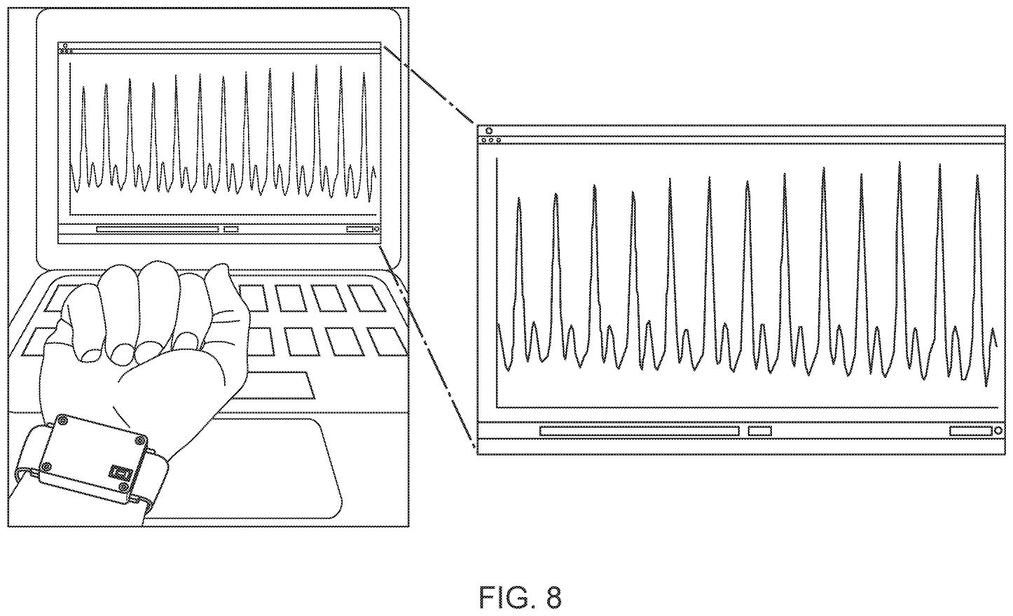

FIG. 10 illustrates an example flow diagram of a method, according to certain example embodiments. In an example embodiment, the method of FIG. 10 may be performed by a blood glucose monitoring system illustrated in FIGS. 1 and 3-9.

According to certain example embodiments, the method of FIG. 10 may include, at 1000, continuously capturing a photoplethysmography signal of a patient in real-time. The method may also include, at 1005, inputting a blood pressure measurement, demographic information, and the photoplethysmography signal into a mobile application. The method may further include, at 1010, transmitting, as input information, the blood pressure measurement, the demographic information, and the photoplethysmography signal to a cloud server. In addition, the method may include, at 1015, generating, based on the input information, a glucose prediction of the patient.

According to certain example embodiments, the input information may be transmitted to the cloud server at every 10 seconds. According to some example embodiments, the glucose prediction of the patient may be generated at every 10 seconds. According to other example embodiments, the photoplethysmography signal of the patient may be captured by a measurement device. According to further example embodiments, the photoplethysmography signal may be captured non-invasively.

In certain example embodiments, the method may also include classifying, based on the glucose prediction, a glucose level of the patient as a warning, as normal, or as abnormal. In some example embodiments, the method may further include providing an alert based on the classification. In other example embodiments, the transmission of the input information to the cloud server may include transmitting the input information to a machine learning model to generate the glucose prediction.

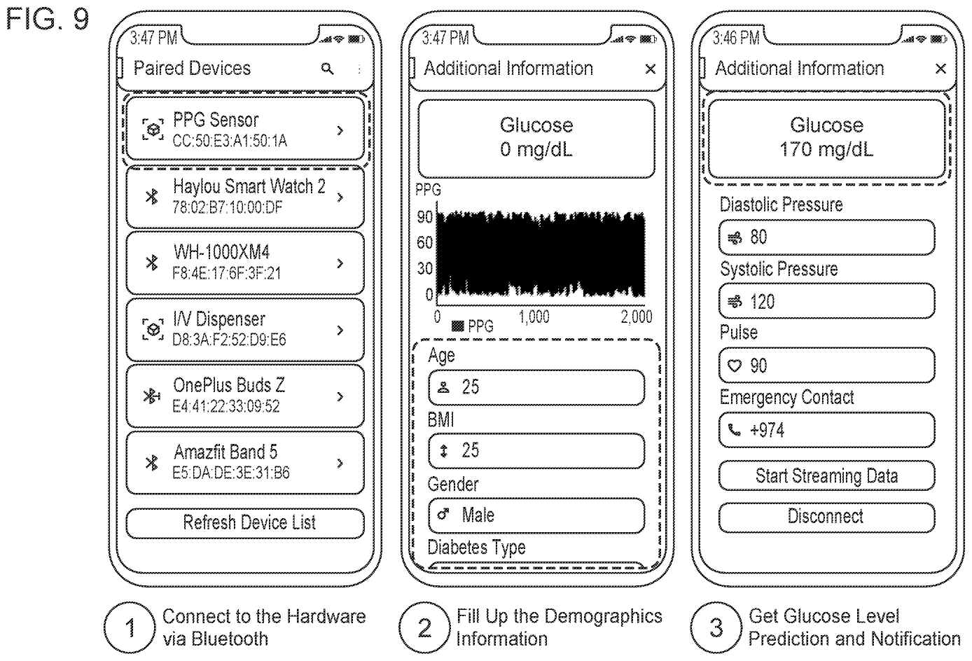

FIG. 11 illustrates an apparatus 10 according to certain example embodiments. In certain example embodiments, apparatus 10 may be a CGM device, a computer, mobile computing device, network device, server, or other similar device. In some embodiments, apparatus 10 may be in communication (i.e., connected to via wired or wirelessly) with other similar computer devices forming a network of connected computer devices.

In some example embodiments, apparatus 10 may include one or more processors, one or more computer-readable storage medium (for example, memory, storage, or the like), one or more radio access components (for example, a modem, a transceiver, or the like), and/or a user interface.

As illustrated in the example of FIG. 12 apparatus 10 may include or be coupled to a processor 12 for processing information and executing instructions or operations. Processor 12 may be any type of general or specific purpose processor. In fact, processor 12 may include one or more of general-purpose computers, special purpose computers, microprocessors, digital signal processors (DSPs), field-programmable gate arrays (FPGAs), application-specific integrated circuits (ASICs), and processors based on a multi-core processor architecture, as examples. While a single processor 12 is shown in FIG. 12, multiple processors may be utilized according to other example embodiments. For example, it should be understood that, in certain example embodiments, apparatus 10 may include two or more processors that may form a multiprocessor system (e.g., in this case processor 12 may represent a multiprocessor) that may support multiprocessing. According to certain example embodiments, the multiprocessor system may be tightly coupled or loosely coupled (e.g., to form a computer cluster).

Processor 12 may perform functions associated with the operation of apparatus 10 including, as some examples, precoding of antenna gain/phase parameters, encoding and decoding of individual bits forming a communication message, formatting of information, and overall control of the apparatus 10, including processes illustrated in FIGS. 1-10.

Apparatus 10 may further include or be coupled to a memory 14 (internal or external), which may be coupled to processor 12, for storing information and instructions that may be executed by processor 12. Memory 14 may be one or more memories and of any type suitable to the local application environment, and may be implemented using any suitable volatile or nonvolatile data storage technology such as a semiconductor-based memory device, a magnetic memory device and system, an optical memory device and system, fixed memory, and/or removable memory. For example, memory 14 can be comprised of any combination of random access memory (RANI), read only memory (ROM), static storage such as a magnetic or optical disk, hard disk drive (HDD), or any other type of non-transitory machine or computer readable media. The instructions stored in memory 14 may include program instructions or computer program code that, when executed by processor 12, enable the apparatus 10 to perform tasks as described herein.

In certain example embodiments, apparatus 10 may further include or be coupled to (internal or external) a drive or port that is configured to accept and read an external computer readable storage medium, such as an optical disc, USB drive, flash drive, or any other storage medium. For example, the external computer readable storage medium may store a computer program or software for execution by processor 12 and/or apparatus 10 to perform any of the methods illustrated in FIGS. 1-10.

In some example embodiments, apparatus 10 may also include or be coupled to one or more antennas 15 for receiving a downlink signal and for transmitting via an uplink from apparatus 10. Apparatus 10 may further include a transceiver 18 configured to transmit and receive information. The transceiver 18 may also include a radio interface (e.g., a modem) coupled to the antenna 15. The radio interface may include other components, such as filters, converters signal shaping components, and the like, to process symbols, carried by a downlink or an uplink.

For instance, transceiver 18 may be configured to modulate information on to a carrier waveform for transmission by the antenna(s) 15 and demodulate information received via the antenna(s) 15 for further processing by other elements of apparatus 10. In other example embodiments, transceiver 18 may be capable of transmitting and receiving signals or data directly. Additionally or alternatively, in some example embodiments, apparatus 10 may include an input and/or output device (I/O device). In certain example embodiments, apparatus 10 may further include a user interface, such as a graphical user interface or touchscreen.

In certain example embodiments, memory 14 stores software modules that provide functionality when executed by processor 12. The modules may include, for example, an operating system that provides operating system functionality for apparatus 10. The memory may also store one or more functional modules, such as an application or program, to provide additional functionality for apparatus 10. The components of apparatus 10 may be implemented in hardware, or as any suitable combination of hardware and software.

According to certain example embodiments, processor 12 and memory 14 may be included in or may form a part of processing circuitry or control circuitry. In addition, in some example embodiments, transceiver 18 may be included in or may form a part of transceiving circuitry.

As used herein, the term “circuitry” may refer to hardware-only circuitry implementations (e.g., analog and/or digital circuitry), combinations of hardware circuits and software, combinations of analog and/or digital hardware circuits with software/firmware, any portions of hardware processor(s) with software (including digital signal processors) that work together to cause an apparatus (e.g., apparatus 10) to perform various functions, and/or hardware circuit(s) and/or processor(s), or portions thereof, that use software for operation but where the software may not be present when it is not needed for operation. As a further example, as used herein, the term “circuitry” may also cover an implementation of a hardware circuit or processor (or multiple processors), or portion of a hardware circuit or processor, and its accompanying software and/or firmware.

In certain example embodiments, apparatus 10 may be controlled by memory 14 and processor 12 to continuously capture a photoplethysmography signal of a patient in real-time. Apparatus 10 may also be controlled by memory 14 and processor 12 to input a blood pressure measurement, demographic information, and the photoplethysmography signal into a mobile application. Apparatus 10 may further be controlled by memory 14 and processor 12 to transmit, as input information, the blood pressure measurement, the demographic information, and the photoplethysmography signal to a cloud server. In addition, apparatus 10 may be controlled by memory 14 and processor 12 to generate, based on the input information, a glucose prediction of the patient.

In some example embodiments, an apparatus (e.g., apparatus 10) may include means for performing a method, a process, or any of the variants discussed herein. Examples of the means may include one or more processors, memory, controllers, transmitters, receivers, sensors, and/or computer program code for causing the performance of the operations.

Certain example embodiments may further be directed to an apparatus that includes means for performing any of the methods described herein including, for example, means for continuously capturing a photoplethysmography signal of a patient in real-time. The apparatus may also include means for inputting a blood pressure measurement, demographic information, and the photoplethysmography signal into a mobile application. The apparatus may further include means for transmitting, as input information, the blood pressure measurement, the demographic information, and the photoplethysmography signal to a cloud server. In addition, the apparatus may include means for generating, based on the input information, a glucose prediction of the patient.

Certain example embodiments described herein provide several technical improvements, enhancements, and/or advantages. In some example embodiments, it may be possible to provide a device that is a non-invasive continuous blood glucose monitoring system. The device may use physiological signal(s) collected non-invasively from the wrist of a user. With the CGM device may continuously estimate blood glucose levels non-invasively and in real-time from PPG signals collected from wristband sensors along with blood pressure and demographic information. The CGM device may also classify a patient's blood glucose level as a warning, normal, or abnormal to aid in daily care at home. In other example embodiments, the CGM device may alert the user/patient through a mobile application executed on a mobile computer device. For example, the mobile application may send a message to an emergency number provided by the user/patient for cases such as extremely high blood sugar (hyperglycemia) or dangerously low blood sugar (hypoglycemia) which may result in a diabetic coma.

As described herein, a computer program product may include one or more computer-executable components which, when the program is run, are configured to carry out some example embodiments. The one or more computer-executable components may be at least one software code or portions of it. Modifications and configurations required for implementing functionality of certain example embodiments may be performed as routine(s), which may be implemented as added or updated software routine(s). Software routine(s) may be downloaded into the apparatus.

As an example, software or a computer program code or portions of code may be in a source code form, object code form, or in some intermediate form, and may be stored in some sort of carrier, distribution medium, or computer readable medium, which may be any entity or device capable of carrying the program. Such carriers may include a record medium, computer memory, read-only memory, photoelectrical and/or electrical carrier signal, telecommunications signal, and software distribution package, for example. Depending on the processing power needed, the computer program may be executed in a single electronic digital computer or it may be distributed amongst a number of computers. The computer readable medium or computer readable storage medium may be a non-transitory medium.

In other example embodiments, the functionality may be performed by hardware or circuitry included in an apparatus (e.g., apparatus 10), for example through the use of an application specific integrated circuit (ASIC), a programmable gate array (PGA), a field programmable gate array (FPGA), or any other combination of hardware and software. In yet another example embodiment, the functionality may be implemented as a signal, a non-tangible means that can be carried by an electromagnetic signal downloaded from the Internet or other network.

According to certain example embodiments, an apparatus, such as a node, device, or a corresponding component, may be configured as circuitry, a computer or a microprocessor, such as single-chip computer element, or as a chipset, including at least a memory for providing storage capacity used for arithmetic operation and an operation processor for executing the arithmetic operation.

One having ordinary skill in the art will readily understand that the invention as discussed above may be practiced with steps in a different order, and/or with hardware elements in configurations which are different than those which are disclosed. Therefore, although the invention has been described based upon these example embodiments, it would be apparent to those of skill in the art that certain modifications, variations, and alternative constructions would be apparent, while remaining within the spirit and scope of example embodiments.

We claim:

1. A method for monitoring blood glucose, comprising:
continuously capturing a photoplethysmography signal of a patient in real-time;
inputting a blood pressure measurement, demographic information, and the photoplethysmography signal into a mobile application;
transmitting, as input information, the blood pressure measurement, the demographic information, and the photoplethysmography signal to a cloud server; and
generating, based on the input information, a glucose prediction of the patient.

2. The method for monitoring blood glucose according to claim 1,
wherein the input information is transmitted to the cloud server at every 10 seconds, and
wherein the glucose prediction of the patient is generated at every 10 seconds.

3. The method for monitoring blood glucose according to claim 1,
wherein the photoplethysmography signal of the patient is captured by a measurement device, and
wherein the photoplethysmography signal is captured non-invasively.

4. The method for monitoring blood glucose according to claim 1, further comprising:
classifying, based on the glucose prediction, a glucose level of the patient as a warning, as normal, or as abnormal.

5. The method for monitoring blood glucose according to claim 4, further comprising:
providing an alert based on the classification.

6. The method for monitoring blood glucose according to claim 1, wherein the transmission of the input information to the cloud server comprises transmitting the input information to a machine learning model to generate the glucose prediction.

7. A blood glucose monitoring system, comprising:
a monitoring device comprising a biological sensor and microcontroller configured to continuously capture a photoplethysmography signal of a patient in real-time;
a user equipment in communication with the monitoring device, wherein the user equipment is configured to receive, as input information, a blood pressure measurement, demographic information, and the photoplethysmography signal; and
a server in communication with the user equipment, wherein the server is configured to generate, based on the input information, a glucose prediction of the patient.

8. The blood glucose monitoring system according to claim 7,
wherein the input information is received by the server at every 10 seconds, and
wherein the glucose prediction of the patient is generated at every 10 seconds.

9. The blood glucose monitoring system according to claim 7, wherein the photoplethysmography signal is captured non-invasively.

10. The blood glucose monitoring system according to claim 7, wherein the user equipment is configured to classify, based on the glucose prediction, a glucose level of the patient as a warning, as normal, or as abnormal.

11. The blood glucose monitoring system according to claim 10, wherein the user equipment is configured to provide an alert based on the classification.

12. The blood glucose monitoring system according to claim 7, wherein the glucose prediction is generated via a machine learning model implemented in the server.

13. A computer program embodied on a non-transitory computer readable medium, the computer program comprising computer executable code which, when executed by a processor, causes the processor to:
continuously capture a photoplethysmography signal of a patient in real-time;
input a blood pressure measurement, demographic information, and the photoplethysmography signal into a mobile application;
transmit, as input information, the blood pressure measurement, the demographic information, and the photoplethysmography signal to a cloud server; and
generate, based on the input information, a glucose prediction of the patient.

14. The computer program according to claim 13,
wherein the input information is transmitted to the cloud server at every 10 seconds, and
wherein the glucose prediction of the patient is generated at every 10 seconds.

15. The computer program according to claim 13, wherein the photoplethysmography signal is captured non-invasively.

16. The computer program according to claim 13, wherein when the computer executable code is executed by the processor, the processor is further caused to:
classify, based on the glucose prediction, a glucose level of the patient as a warning, as normal, or as abnormal.

17. The computer program according to claim 16, wherein when the computer executable code is executed by the processor, the processor is further caused to:
provide an alert based on the classification.

18. The computer program according to claim 13, wherein the transmission of the input information to the cloud server comprises transmitting the input information to a machine learning model to generate the glucose prediction.
